(19) United States
(12) Patent Application Publication
(10) Pub. No.: US 2009/0143864 A1
(43) Pub. Date: Jun. 4, 2009

(54) Process for manufacturing a prosthetic joint

(76) Inventors: Roelof Marissen, Born (NL); Leo Smit, Oirsbeek (NL); Carina S. Snijder, Sittard (NL)

Correspondence Address: NIXON & VANDERHYE, PC, 901 NORTH GLEBE ROAD, 11TH FLOOR, ARLINGTON, VA, 22203, US

(21) Appl. No.: 10/584,755

(22) PCT Filed: Jan. 7, 2005
(86) PCT No.: PCT/NL05/00005

§ 371 Date: Nov. 29, 2006

(30) Foreign Application Priority Data
Jan. 7, 2004 (NL) 1025/76

(51) Int. Cl. A61F 2/30; B29C 70/04

(52) U.S. Cl. 623/18.11; 264/258

(57) ABSTRACT

The application relates to a process for manufacturing a prosthetic joint with at least one loaded surface which consists at least partially of polyethylene, comprising compressing one or more layers of a woven fabric of drawn gel-spun polyethylene fibres into the desired shape in a hollow mould part using a plug at a pressure of at least 0.05 MPa and at a temperature of between 120 and 165° C. and below the crystalline melting point of the polyethylene at the prevailing temperature and pressure, without a matrix material being present, and at least the woven fabric in a layer situated on a loaded surface comprising at least 90 wt % of fibres with a titre of at most 1000 denier, and to a prosthetic joint with a crease-free surface.

The invention relates to a process for manufacturing a prosthetic joint with at least one loaded surface, in particular a loaded surface that is curved in one or more directions, which surface consists at least partially of polyethylene. Polyethylene and in particular ultra-high molecular weight polyethylene (UHMWPE) is a known and frequently applied material in the manufacture of prosthetic or replacement joints. The biological inertia and the high wear resistance make the material very suitable for internal application in mammals, esp. humans. The application in prosthetic joints, in particular in the loaded parts thereof, is known. In particular the inside of joint sockets, which when loaded come into contact with the joint balls moving therein and usually made of metal, are an example hereof, as are components of artificial knee, hip, elbow, shoulder, wrist, ankle, toe and finger joints.

Suitable UHMWPE is that with an intrinsic viscosity (IV, measured on a solution in decalin at 135° C.) of between 4 and 40 dl/g, preferably between 12 and 30 or even 15 and 25 dl/g. Preferably the UHMWPE is a linear polyethylene with less than one side chain per 100 carbon atoms and preferably less than one side chain per 300 carbon atoms, with a side chain or branch usually containing at least 10 carbon atoms. The linear polyethylene may further contain up to 5 mol % of one or more comonomers, for example, alkenes such as propylene, butene, pentene, 4-methylpentene or octene.

The UHMWPE may contain a small quantity of relatively small groups as side chains, preferably a C1-C4 alkyl group. In that case the UHMWPE preferably contains methyl or ethyl side chains and more preferably methyl side chains. Their number then preferably is 0.2-10, more preferably 0.3-5 per 1000 carbon atoms. Also mixtures of different types of UHMWPE that differ in terms of for example IV, molecular weight distribution and/or the number of side chains can be applied in the process according to the invention. The PE part of the prosthetic joint can be directly anchored to bone, either mechanically or using bone cement, with an intermediate layer of another polymer, for example PMMA, optionally being present. From WO 00/59701 it is known to manufacture the said part by compressing UHMWPE powder at elevated temperature and at elevated pressure to form a block from which the part with the desired shape is machined.

A meanwhile commonly known problem with this application of UHMWPE, despite its high wear resistance, is the release during use of polyethylene particles as a consequence of the cooperating joint parts moving along each other. In particular particles with a size of between 0.5 and 10 μm are found to result in biological reactions in the human body, which can lead to functional loss of the surrounding bone and inflammation reactions of the body.

The invention now aims to provide a process that does not entail or entails to a lesser extent said disadvantage.

This object is achieved according to the invention by compressing in a mould to a desired shape, between a male mould part, further called plug, and a female hollow mould part, one or more layers of a woven fabric of drawn gel-spun polyethylene fibres at a pressure of at least 0.05 MPa and at a temperature between 120 and 165° C. and below the crystalline melting point of the polyethylene at the prevailing temperature and pressure, without a matrix material being present, and at least the woven fabric in a layer situated on a loaded surface comprising at least 90 wt % of polyethylene fibres with a titre of at most 1000 denier.

Surprisingly, it was found that polyethylene in a thus manufactured prosthesis releases significantly less particles during use, in particular in the above-mentioned range, which particles may result in undesirable reactions in the human body, than from the polyethylene in the known prostheses. The gel spinning process described hereafter, which the fibres have as their prior history, is found to impart special properties on the surface of the compressed woven fabric obtained by the process according to the invention, which properties deviate from those of the surface of an object that is moulded from powder and then machined. This lengthens the service life of the prosthesis and prevents early replacement operations that are expensive and painful for the patient.

A further advantage of the process according to the invention is the low creep of the obtained prosthesis, which assures long-term retention of the fit on the complementary, cooperating joint part. Furthermore, the surface of the prosthesis needs no further operations, in contrast with the known process where the desired shape is obtained by machining. The latter leads to greater surface roughness and a greater risk of particles being released from the surface than with the process according to the invention.

A loaded surface is herein understood to be a surface that is exposed to mechanical loading during use of the prosthesis after implantation in the human body.

With the process according to the invention a compact part is obtained without application of a separate matrix material to attach the fibres to each other or fill up the voids between them. The presence of a matrix material may have the possible disadvantage that when the joint is loaded it may release particles whose size is within the biologically dangerous range.

The process according to the invention starts from a woven fabric of drawn gel-spun polyethylene fibres. Such fibres are known per se, as are the processes for the production thereof. Essential steps in the manufacture of such fibres are dissolving the polyethylene in a solvent, spinning the solution through a spinneret with several holes to form fibres consisting of the solution, solidifying the fibres by cooling to below the dissolving point of the solution or other techniques known in fibre spinning, drawing the cooled fibres in one or more steps at a temperature below, but preferably near, the crystalline melting point of the fibres at the prevailing temperature and imposed drawing tension, if they no longer contain any solvent or the dissolving temperature, if the fibres still contain solvent. The solvent is removed before, during or after drawing so that finally at least virtually solvent-free fibres are obtained. In the fibres thus obtained, as a consequence of drawing, a large proportion of the PE is molecularly oriented. This proportion is found to substantially contribute to the favourable properties of the fibres. As a rule, a small proportion is less molecularly oriented and is found to have a lower melting point than the molecularly oriented part. This unique property of drawn, gel-spun polyethylene fibres makes the said fibres especially suitable for application in the process according to the invention.

Examples of thus obtained fibres, hereafter referred to as gel fibres, are UHMWPE fibres commercially available the under the trade names of Dyneema® and Spectra®. Fibre is here understood to be especially a multifilament yarn that consists of a number of, for example of 2 to 2000, monofilaments.

The fibres are applied in the form of a woven fabric, which here also includes a knitted fabric. A knitted fabric is here understood to be a sheet-shaped fibrous structure wherein the fibres have obtained, by various forms of entanglement, a certain measure of cohesion. In a woven fabric each fibre runs alternately over and under one or more crossing fibres and thereby appears and disappears in a regular pattern on and from the surface. The length of the fibre part appearing on the surface between two successive places where a fibre runs over a crossing fibre is called the exposed fibre length.

It has been found that the exposed fibre length on the surface of the fibres or yarns in the woven fabric has a significant effect on the wear properties of the manufactured prosthesis. This exposed fibre length depends on the yarn titre and the way in which the fibres in the woven fabric cross each other. For example, in a 1-over-1 woven fabric, in both intersecting directions, each fibre runs alternately over and under the fibres laid successively next to each other in the crossing direction. In a 1-over-2 woven fabric, each fibre runs in one direction alternately over and under a pair of two adjacent fibres in the crossing direction. In a 2-over-2 woven fabric, the latter is the case in both directions. It has now been found that i-over-j woven fabrics, wherein both i and j are ≦3, yield prostheses with a good wear resistance in the process according to the invention. Very good results are achieved when i and j are ≦2 and the best results are achieved when either i or j is at most 2 and the other is equal to 1. The so-called plain weave, wherein i and j are both 1, is most preferred.

In addition to the fabric's weave pattern, its density also has an effect on the exposed fibre length of the fibres on the surface of the woven fabric. This density is preferably high, with the yarn titre being a limiting factor. Where fibres with a titre of t denier are applied in an i-over-j fabric, the fibre density n, that is, the number of fibres per cm on the surface, preferably is at least 250/√t cm⁻¹, more preferably at least 330/√t and most preferably at least 350/√t cm⁻¹. The corresponding exposed fibre length m on the surface of an i-over-j woven fabric of fibres with a titre of t denier is preferably at most √t/(250/max(i,j)) cm, more preferably at most √t/(330/max(i,j)) and most preferably at most √t/(350/max(i,j)) cm, wherein max(i, j) is the greater of i and j.

Said values apply to the fabric before compression. If a multi-layered fabric is applied, said values apply at least to the fabric lying in a loaded surface of the prosthesis. Analogous considerations apply for a knitted fabric. For the woven fabric layers not in a loaded surface lower fibre density values are permissible. Furthermore, it was found that the quantity of abraded particles in the range from 0.5 to 10 μm as a consequence of joint movements is relatively lower when the fibres consist of monofilaments with a titre of between 0.5 and 10 denier per filament (dpf) and preferably between 1 and 5 dpf. The fibres themselves, which consist of multiple monofilaments, preferably have a fibre titre of at least 10, preferably at least 20 and more preferably at least 40 denier and of at most 1000, preferably at most 900, more preferably at most 750 or even 500 denier, in view of the same advantageous effect.

Furthermore it was found that the fibre density can be increased, and so the exposed fibre length can be reduced, when the woven fabric is subjected to a heat treatment under tension before compression. The applied tension must be adequate to permit some shrinkage, but care should be taken here to prevent the woven fabric from creasing or bubbling. Suitable temperatures are those between 120 and 145° C., but in any case below the crystalline melting point of the polyethylene at the prevailing temperature and tension. Normally, maintaining the temperature and tension for a period of between 1 and 30 minutes is adequate to accomplish substantial increase in density of the woven fabric. Preferably the fibre density after the heat treatment is at least 360/√t or even at least 380/√t cm⁻¹.

The woven fabric may consist of a single layer, a single-layer woven fabric, but the woven fabric preferably consists of several layers stacked on each other, a multi-layered fabric. The woven fabric may also be a three-dimensional (3D) woven or knitted construction. This has the advantage that the woven fabric has no visible fibre ends that might require further finishing. A combination of single and multi-layered fabrics may also be applied in multi-layered fabric. Combinations of woven fabrics and knitted fabrics can also be applied. A multi-layer or 3D construction can be stitched through, preferably with a fine thread, preferably without threads being introduced with a larger exposed fibre length than those of the woven fabric on the surface. It invariably holds that the requirements specified for the fibre density n and the corresponding exposed fibre length values m apply to at least 90% and preferably at least 98% to even 100% of the woven fabric or knitted fabric situated on the loaded surface. The woven fabrics not directly situated on the loaded surface of the prosthesis may possess lower n or higher m values.

The woven fabric is compressed into the desired shape. This shape is determined by the joint part to be replaced by the prosthesis. The surface facing the complementary joint part, for example that of a hip socket, will have a shape that corresponds with the surface of the cooperating complementary joint part, in this case the ball of the part of the hip joint connected to the thighbone. The opposite surface of the prosthesis faces the body and is arranged such that it can be connected to the body. To that end, a metal or plastic structure suitable to be attached to the body may be provided in the hollow mould part. During compression the woven fabric can then adhere thereto, either directly under influence of compression or by means of adhesives. The process according to the invention in that case directly provides a prosthesis that can be fixed in the body, for example mechanically or by means of a bone cement or resin known per se. In another embodiment the inside of the hollow mould part is unlined and the process according to the invention provides only an UHMWPE layer which is yet to be attached to a structure which is suitable to be attached to the body. Techniques for attaching a prosthesis to the body are known per se and do not form part of this invention.

The woven fabric is compressed into the desired shape in a mould using corresponding plug and hollow mould parts. The surface of the plug, which during compression comes into contact with the woven fabric, has the shape required of the surface of the cooperating complementary part in the joint. The inside surface of the hollow mould part is preferably adapted to the shape of the plug and the desired shape of the prosthesis such that, as the woven fabric is compressed, the resulting layer has a desired thickness distribution and the desired shape. The desired layer thickness may be equal throughout the surface but it may also be preferred to have a greater thickness in some places than in other places in connection with the future loading during operation of the relevant joint. Thickness variations can be provided by locally applying more or thicker layers. If a three-dimensional woven fabric is applied, the desired thickness variations can already be provided during weaving. Local thickness variations can be applied to adapt the mechanical behaviour to the mechanical loads in localised areas. A locally greater thickness imparts greater flexural rigidity and strength in a localised area. This allows better load transmission to a metal support structure or even directly to the bone to be achieved.

Compression takes place at elevated temperature and pressure. The temperature at which compression takes place at the applied pressure should be within in a range where only a part of the UHMWPE in the woven fabric melts or can flow under that pressure. The size of this part is determined by the requirement that on the one hand sufficient material melts or becomes liquid to obtain the desired density after compression and on the other hand sufficient material remains in the oriented state to effectively retain the properties of the original fibres. For highly drawn UHMWPE fibres this temperature typically is between 135° C. en 165° C. With increasing temperature the pressure will also need to be chosen higher so as to prevent the fibres from melting completely. A very high pressure is also needed at temperatures at the lower end of the specified range in combination with a longer pressing time to achieve adequate compaction. With the above guidelines one skilled in the art can determine by routine experiments suitable combinations of pressing temperature, pressing pressure and pressing time to achieve adequate compaction. The prosthesis can be also pressed in a number of steps at different pressures and temperatures.

The flowing or molten part of UHMWPE, under the influence of the applied pressing pressure, ensures that voids in the woven fabric are filled and that a, preferably smooth, surface is formed corresponding to that of the plug. The surfaces of the plug and of the hollow mould part are chosen in such a way that a surface is formed on the prosthesis with the desired surface characteristics and in general as smooth as possible.

The temperature should remain so low that the part of the UHMWPE in the fibres that is molecularly oriented by drawing retains this orientation in at least a considerable measure in order to retain the favourable wear properties. Preferably, the initial flexural modulus of the compressed woven fabric in the prosthesis, measured according to ASTM D790M on a fabric sample having a length over thickness ratio of at least 32, is at least 20% of that of the fibres in the starting material. If necessary for obtaining said ratio layers in the fabric far removed from the surface can be peeled off. The pressure with which the woven fabric is compressed into the desired shape should be at least so large that the woven fabric becomes a compacted unitary part, which means that the molten part of the UHMWPE completely or almost completely fills the voids in the woven fabric. On or in the woven fabric there may be present for example a substance with a medicinal effect or with a contrasting effect for X-ray radiation or with the usual scanning techniques. These do not, however, have any function in compacting the woven fabric package. Such additives should be sufficiently resistant to the applied pressing temperatures so as to be able to still serve the intended function in the ready-made prosthesis. A measure of the amount of voids that is present in a compacted woven fabric is the density of the compacted woven fabric. This is preferably at least 90% of the density of the UHMWPE from which the fibres have been manufactured and preferably at least 95 and even 98% or 99% thereof. The pressure amounts therefore to at least 0.05 MPa. Pressures up to 100 and even 200 MPa are permissible, where the pressing time can be shorter with increasing pressing pressure. In general, a higher pressure gives better results, the applied pressure is thus preferably at least 0.5, 1, 5, 10, 25, or even at least 50 MPa. Basically, the applicable pressing pressure is only limited by the available equipment. The fibre material can in fact withstand any realistically attainable pressure. Pressures up to for example 100 MPa or even 200 MPa and even higher can be applied for the fibre material without objection. Also, at elevated pressure it is possible to use a lower pressing temperature to achieve the desired density. On the other hand, at elevated pressure the temperature at which the molecular orientation is lost is higher. A combination of high pressure and high temperature makes the required pressing time shorter. In general, it is advantageous to keep the total temperature load, which is determined by the temperature level and the time during which it is applied, low to prevent as much as possible degradation of the polyethylene and deterioration of the properties acquired by drawing.

The elevated pressure and temperature should be maintained long enough to achieve the desired compaction, that is, the filling of the voids between the fibres with the molten or flowing material that is unoriented or of low molecular orientation. The required combination of pressure, temperature and time can be established by simple experimentation by in each case determining the density of the obtained compacted woven fabric and the modulus thereof. If desired, compaction may be carried out by successively using different combinations of pressure and temperature.

A suitable process for compressing fibrous structures that may be applied for compressing the woven fabric in the process according to the invention is that disclosed in U.S. Pat. No. 5,628,946. In that document it is described how a wide variety of fibre structures such as uniaxially aligned or twisted bundles of fibres, staple fibres in a mat, woven bundles and crossed layers of parallel fibre bundles, which may all consist of a large variety of polymers, can be compacted in order to obtain an object with good mechanical properties. The insight that a prosthesis can be manufactured, from which few particles are released with a size harmful for the human body, by compressing specifically a woven fabric of gel-spun UHMWPE fibres is, however, lacking completely in this document.

Another suitable process for compressing fibrous structures that may be applied to compress the woven fabric of the process according to the invention is that disclosed in U.S. Pat. No. 6,482,343. In this document it is described how diverse physical forms of polymers, such as powders, granules, a tape, fibres, disks, rings and the like, which may consist of a large variety of polymers, can be compacted to obtain an object with good mechanical properties. The insight that a prosthesis can be manufactured, from which few particles are released with a size harmful for the human body, by compressing specifically a woven fabric of gel-spun UHMWPE fibres is lacking completely in this document.

A disadvantage of the known, suitable processes for pressing fabric into the desired shape in a mould is that creasing can occur on the surface during pressing of flat woven fabrics into three-dimensional shapes. Creasing can in particular occur already in case of small deformation if the densely woven fabrics, preferably applied in the process according to the invention, are used. This creasing is highly undesirable in a prosthetic joint, because, owing to the sliding movement of the cooperating joint parts relative to each other, the crease may peel off in the longer term and move partially or even completely freely between these parts. Such movement will cause serious wear and perhaps even blocking of the joint. Creasing should therefore be prevented.

A further object of the process according to the invention is therefore also to provide a process for manufacturing a prosthetic joint, in particular a prosthetic joint curved in one or more directions, with a crease-free loaded surface from woven fabrics with a high density, in particular from woven fabrics with a fibre density at least 250/√t or even at least 330/√t or at least 350/√t cm⁻¹, or otherwise expressed, an exposed fibre length of the fibres on the surface of at most √t/(250/max(i,j)) or at most √t/(330/max(i,j)) or even at most √t/(350/max(i,j)) cm.

It has been found that creasing on the surface can be completely or almost completely prevented when the process comprises tensioning the woven fabric at a temperature between 0 and 5° C. lower than the temperature at which compression takes place, bringing the woven fabric brought to the required temperature into contact with the hollow mould part under pressure of the plug for a period of between 1 and 30 minutes and compressing the woven fabric under a pressure of at least 0.05 MPa for a period of between 2 and 30 minutes at a temperature of between 120 and 165° C. and below the crystalline melting point of the polyethylene at the prevailing temperature and pressure.

In this process a part of the woven fabric is found to be elongated under the tension applied for contacting the woven fabric with the hollow mould part at elevated temperature, which elongation prevents creasing.

A possible explanation for this is that, under the applied conditions, further drawing occurs with retention or even improvement of other properties of PE fibres, in particular of gel-spun UHMWPE fibres, which are accordingly preferably applied in the present process.

This preferred process is advantageous in particular for making prostheses that comprise shapes with a relatively small radius of curvature, such as hip sockets, but can also be applied advantageously for prostheses with less curved or arched surfaces.

In one embodiment of the present process a fabric package is used which is larger than needed for the dimensions of the prosthesis to be manufactured. On positioning this package in or over the mould opening, a part will protrude outside the opening. This protruding part is immobilised for example by pressing it against the outside surface of the mould. The pressure should be so high that the immobilised woven fabric cannot or can only to a negligible extent slip away when the plug presses the woven fabric into shape in the hollow mould.

In one embodiment, the woven fabric, or fabric package, is laid over the mould opening, with the mould having been preheated to a temperature 0 to 5° C. below that at which the final compacting will take place but high enough to make the fabric package adequately mouldable in the steps described hereafter wherein the woven fabric in the desired shape is completely contacted with the hollow mould part and plug. To that effect a ring-shaped element is forcibly pressed onto the part of the woven fabric protruding outside the mould opening and resting on the mould body. The ring-shaped element is preferably also preheated to a temperature in the range indicated above for the mould. The contact force is high enough to cause such friction that the fibres of the woven fabric will not or scarcely shift under the ring-shaped element during the following process steps. Next, the plug, also preheated to a temperature in said range, is brought into contact with the woven fabric in order to bring it to the desired temperature. To obtain good contact, the plug is pushed down until the fibres are slightly tensioned. The applied tension is high enough to prevent relaxation of the reinforced chains in the fibres of the woven fabric at the plug temperature. The resultant elongation should be less than the elongation at break under the prevailing conditions so as to prevent fibre breakage. This condition is maintained until the fabric package has at least virtually reached the plug temperature, in any case high enough to make the fabric package adequately mouldable for the following shaping step. The heating process can be accelerated by additionally adding heat to the fabric in another way than via contact with the plug, for example by using heated air. The surface temperature of the fabric package should, however, remain below the temperature at which the above requirements concerning relaxation and melting can no longer be met. In that next step the plug is moved further down at such a speed that the plug and mould with the woven fabric in-between them make full contact after a time of between 1 or 2 and 30 minutes. A suitable time can be determined by simple experimentation and is dependent on for example the molecular weight of the polyethylene in the fibres and the temperature of the mould and the plug. The drawing rate of the fibres during this step is preferably between 0.0009 and 0.025 sec⁻¹ and more preferably between 0.001 and 0.02 sec⁻¹. In this phase, too, fibre breakage should be prevented as much as possible. During this time the fibres are elongated under tension as a result of creep deformation and further drawing, and a crease-free shape is found to be obtained. After achieving full contact, whereby the woven fabric is contacted with both the plug and hollow mould part over its whole surface, the pressure is raised to the desired pressing pressure. In a simple embodiment, compaction can already be achieved at this pressing pressure if compression is performed for an adequately long time. Preferably the temperature of the hollow mould part and plug is increased to the desired maximum pressing temperature when the pressing pressure has been reached. With increasing pressure a higher maximum temperature can be chosen, without the orientation of the fibres being lost to an unacceptable degree due to melting. These temperature and pressure are maintained, as described above, for the required time. As a rule a time of between 2 and 30 minutes is adequate. Next, the whole assembly of hollow mould part, plug and woven fabric is cooled to well below, for example 20 to 100° C. below, the melting point of the fibres, and the plug is retracted. The pressure is maintained until the sufficiently low temperature has been reached. Finally, the woven fabric is taken from the hollow mould part and cooled to room temperature. The shaped product is crease-free. The density is virtually that of the fibre material, typically more than 98 or 99% or up to even 100% thereof. The edges of the formed prosthesis are finished by removing the protruding part where necessary.

A similarly made product that is not immobilised and has not been elongated by creep deformation admittedly also has a density almost equal to fibre density, but is found to exhibit creases in a number of locations along the surface. These creases extend from the top edge of the product in the wall of the product over approximately 25% of the distance to the deepest part of the product.

When a woven fabric is compressed the cross-section of a fibre, a bundle of filaments, therein will generally show flattening, especially of a fibre at the surface. The cross-section of a fibre is herein described by the ratio of the fibre dimension in the direction perpendicular to the longitudinal fibre direction and parallel to the surface or along its curved surface, and the fibre dimension in the direction perpendicular to the surface. In relatively loose woven fabrics the ratio of said dimensions after compression is typically larger than 20. In a part formed with the process of the present invention from woven fabrics with a high density and correspondingly small exposed fibre length on the surface as defined above, and that would give rise to creasing in a process not according to the invention, this ratio is at most 15. These dimensions can be determined with a microscope after having cut the pressed fabric with a new sharp diamond knife at cryogenic temperatures. It should be noted that the dimensions are measured at the remaining substrate and not on the cut-off slices. In many cases the dimensions at the surface are directly visible in a pattern stemming from the original fabric structure. For this purpose, an optical or electron microscope (e.g. SEM) can be used.

Crease-free prostheses from densely woven fabrics with a small exposed fibre length at the loaded surface, which are crease-forming as such, are not known and therefore the invention also relates to a prosthetic joint with a crease-free loaded surface and formed from one or more layers of woven fabrics of drawn gel-spun polyethylene fibres compressed onto each other, wherein the average ratio of the dimension of a compressed fibre on the loaded surface perpendicular to its longitudinal direction and measured along the surface and the corresponding dimension perpendicular to the surface is at most 15.

Preferably said ratio is at most 9 or even at most 7.5. The density of the prosthesis preferably is at least 98 or 99 to even virtually 100% of the density of the fibre material. It is unexpected that prostheses compressed to such a high density can be manufactured in combination with such a low ratio between said dimensions of the compressed fibre. They exhibit, in addition to being crease-free, also a high wear resistance and very good mechanical properties.

Preferably the polyethylene is UHMWPE. The prosthesis preferably consists of one or more fabric layers compressed onto each other. Further preferences agree with those that are stated above in the description of the process.

The process according to the invention can be applied for manufacturing loaded surfaces of prosthetic joints or sections thereof such as hip sockets, shoulder sockets, tibial trays, the thighbone part of the knee joint, knee caps, and the cooperating complementary parts of finger, wrist, toe and jaw joints.

The described preferred process is elucidated on the basis of the following drawings.

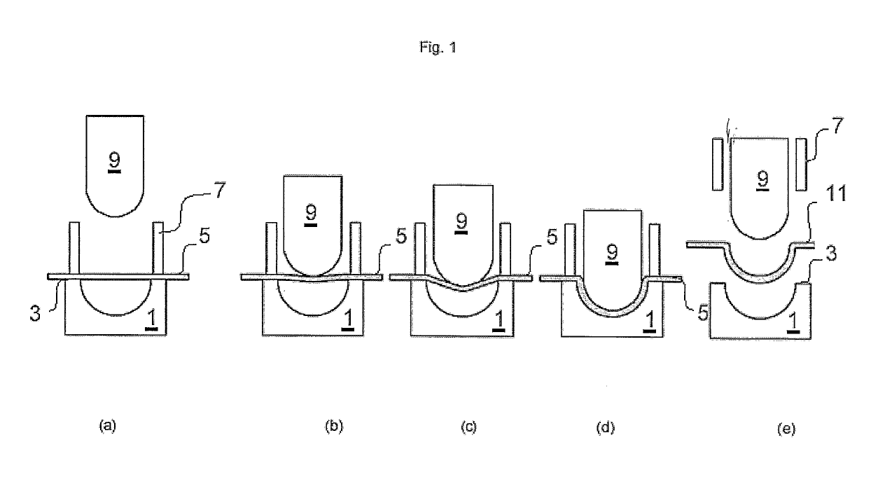

FIG. 1(a) up to and including 1(e) show the successive steps.

FIG. 1(a) shows a socket-shaped mould with top edge 3. On this top edge 3 rests a package 5 consisting of a number of fabric layers. The package 5 is tightly pressed against top edge 3 by means of annular pressure element 7. Plug 9 is free of the package. Parts 1, 3 and 9 have been heated to 135° C.

In FIG. 1(b) plug 9 is in contact with package 7 and presses this package over a small distance downward, so that tension is created in the package. The assembly is held in this condition until the fabric package has about reached the temperature of the plug.

FIG. 1(c) shows how the plug 9 is pressed further down, with the fabric package 5 being pressed further down until in FIG. 1(d) it is clamped between plug 9 and hollow mould part 1. The plug is then pressed on to a pressure of 15 MPa and this condition is maintained for 12 min. Finally, the assembly of hollow mould part, plug, pressure element and woven fabrics is cooled to 80° C. while the applied pressure is maintained, after which the plug is removed.

FIG. 1(e) shows the final condition where the product 11 formed between hollow mould part and plug is removed for further finishing steps.

1. Process for manufacturing a prosthetic joint with at least one loaded surface that consists at least partially of polyethylene, comprising compressing in a mould to a desired shape, between a hollow mould part and a plug, one or more layers of a woven fabric of drawn gel-spun polyethylene fibres at a pressure of at least 0.05 MPa and at a temperature between 120 and 165° C. and below the crystalline melting point of the polyethylene at the prevailing temperature and pressure, without a matrix material being present, and at least the woven fabric in a layer situated on a loaded surface comprising at least 90 wt % of polyethylene fibres with a titre of at most 1000 denier.

2. Process according to claim 1, wherein the woven fabric in a layer on a loaded surface is an i-over-j woven fabric of fibres with a titre t denier with an exposed fibre length on the surface of at most √t(250/max(i,j)) cm.

3. Process according to claim 2, wherein the exposed fibre length on the surface is at most √t/(330/max (i,j)) cm.

4. Process according to claim 3, wherein prior to compression the woven fabric is kept at a temperature of between 120 and 145° C. for a period of between 1 and 30 minutes and under tension.

5. Process according to claim 1, wherein the polyethylene has an IV, measured in decalin at 135° C., of 4-40 dl/g.

6. Process according to claim 1, wherein at least the woven fabric in a layer situated on a loaded surface comprises at least 90 wt % of fibres that consist of monofilaments with a titre of at most 10 denier per filament.

7. Process according to claim 1, wherein at least the woven fabric situated in a layer on a loaded surface is a 1×1 plain weave fabric.

8. Process according to claim 1, wherein the woven fabric is a multi-layered woven fabric.

9. Process according to claim 1, wherein the woven fabric is a three-dimensional woven fabric.

10. Process according to claim 1, comprising bringing the woven fabric, under tension, to a temperature between 0 and 5° C. below the temperature at which compression takes place, contacting the woven fabric brought to the required temperature with the hollow mould part under the pressure of the plug for a period of between 1 and 30 minutes, and compressing the woven fabric under a pressure of at least 0.05 MPa for a period of between 2 and 30 minutes.

11. Process according to claim 10, wherein at least the woven fabric in the layer situated on a loaded surface has an exposed fibre length on the surface of at most √t/(250/max(i,j)) cm.

12. Process according to claim 10, wherein the prosthetic joint is a hip socket.

13. Prosthetic joint with a crease-free loaded surface and formed from one or more layers of woven fabrics of drawn, gel-spun polyethylene fibres compressed onto each other, wherein the average ratio of the dimension of a compressed fibre on the surface perpendicular to its longitudinal direction and measured along the surface and the corresponding dimension perpendicular to the surface is at most 15.

14. Prosthetic joint according to claim 13, wherein said ratio is at most 9.

15. Prosthetic joint according to claim 14, wherein said ratio is at most 7.

16. Prosthetic joint according to claim 13, wherein the IV, measured in decalin at 135° C., of the polyethylene is between 4 and 40 dl/g.
